SEQUENCE LISTING (9 sequences)

SEQ ID NO: 1 - protein, 7 aa, Homo sapiens
GEAGKPG

SEQ ID NO: 2 - protein, 7 aa, Homo sapiens
CGEAGKP

SEQ ID NO: 3 - protein, 9 aa, Homo sapiens
CGEAGKPGK

SEQ ID NO: 4 - protein, 8 aa, homo sapiens
  MOD_RES (7)..(7): 4Hyp
CGEAGKPG

SEQ ID NO: 5 - protein, 7 aa, Homo sapiens
CEAGKPG

SEQ ID NO: 6 - protein, 7 aa, Homo sapiens
CGEGKPG

SEQ ID NO: 7 - protein, 8 aa, Homo sapiens
CGAEGKPG

SEQ ID NO: 8 - protein, 8 aa, Homo sapiens
CGEAGKPG

SEQ ID NO: 9 - protein, 6 aa, homo sapiens
EAGKPG

(19) United States
(12) Patent Application Publication
(10) Pub. No.: US 2011/0281285 A1
(43) Pub. Date: Nov. 17, 2011

(54) Assay for Detecting Cathepsin K Activty in Bone, Cartilage and/or Soft Tissue

(76) Inventors: John Spencer Mort, Pointe-Claire (CA); Anthony Robin Poole, Lancaster (CA); Valeria Mihaela Dejica, Montreal (CA)

(21) Appl. No.: 12/961,280

(22) Filed: Dec. 6, 2010

Related U.S. Application Data
(63) Continuation of application No. 12/764,735, filed on Apr. 21, 2010, now abandoned.
(60) Provisional application No. 61/171,309, filed on Apr. 21, 2009.

(30) Foreign Application Priority Data
Jun. 18, 2009 (CA) 2,669,470

(51) Int. Cl. G01N 33/573; C07K 16/18; G01N 21/64

(52) U.S. Cl. 435/7.92; 435/7.1; 530/387.9

(57) ABSTRACT

There is provided a method for detecting cathepsin K activity in bone, cartilage and/or connective tissue, the method comprising providing a biological sample, contacting the sample with an antibody having binding specificity for a neoepitope comprising EAGKPG (SEQ ID NO: 9), and detecting an immunocomplex formed between the antibody and the neoepitope, wherein presence of the immunocomplex is indicative of cathepsin K activity in bone, cartilage and/or connective tissue. There is also provided a kit for detecting cathepsin K activity in bone, cartilage and/or connective tissue.

### CROSS-REFERENCE TO RELATED APPLICATIONS

The present application claims the benefit under 35 USC §119 of U.S. Provisional Patent Application Ser. No. 61/171,309 filed on Apr. 21, 2009 and of Canadian Patent Application Serial No. 2,669,470 filed on Jun. 18, 2009, the disclosure of each is herein incorporated by reference in their entirety.

### FIELD OF THE INVENTION

This application relates to a method and kit for detecting cathepsin K activity in bone, cartilage and/or soft tissue. In particular, the method and kit comprise use of an antibody having binding specificity for a fragment of collagen resulting from cleavage by cathepsin K.

### BACKGROUND OF THE INVENTION

The following review of the state of the art is merely provided to aid in the understanding of the present invention and neither it nor any of the references cited within it are admitted to be prior art to the present invention.

Degradation of bone and cartilage are pathological consequences of osteoarthritis (OA) and certain types of cancers. Collagen degradation is considered to be a key process in articular cartilage degeneration in OA. Damage to articular cartilage results in the loss of the tensile properties which are determined by the collagen fibrillar network (Kempson G E, Muir H, Pollard C, Tuke M. Biochim Biophys Acta 1973; 297: 456-72). Cartilage swelling and deformation associated with cartilage collagen degradation is one of the hallmarks of early OA (Bank R A, Soudry M, Maroudas A, Mizrahi J, TeKoppele J M. Arthritis Rheum 2000; 43: 2202-10).

Type I collagen (Col I) is the most abundant collagen of the human body. It is present in scar tissue, the end product when tissue heals by repair. It is found, inter alia, in tendons, the endomysium of myofibrils and the organic part of bone, it is found in skin, ligaments, cornea, intervertebral disks, dentine, arteries and granulation tissues (Wardale and Duance, J Cell Sci. 1993 August; 105 Pt 4:975-84; Wardale and Duance, J Cell Sci. 1994 January; 107 Pt 1:47-59). Similarly to other fibrillar collagens this molecule comprises three polypeptide chains (α-chains) which form a unique triple-helical structure. It is a heterotrimer of two α1(I) and one α2(I) chains. Cleavage of Col I by cathepsin K in bone is well accepted. It is currently believed that cleavage of Col I in other connective tissues is mediated at least by collagenases (matrix metalloproteinases 1 and 13).

Type II collagen (Col II), the major structural component of the extracellular matrix of articular cartilage, is composed of three identical α chains that form a triple helix. These molecules self-associate to form the collagen fibrils, which are stabilized by intermolecular crosslinks. In osteoarthritis (OA), the anabolic/catabolic balance of articular cartilage is disrupted with excessive cleavage of Col II involving collagenases, which are matrix metalloproteinases or MMPs.¹⁻⁴ This leads to collagen denaturation^{5,6} and disruption of the collagen fibrillar network with a loss of the tensile properties of articular cartilage.⁷ The collagenases cleave triple helical Col II at a single site (Gly775-Leu/Ile776) toward the C-terminal end, resulting in the release of 3/4N-terminal (TC^{A}) and 1/4C-terminal (TC^{B}) collagen fragments.^{3,8}

Until recently the collagenases were the only proteinases present in connective tissues known to be capable of cleaving the triple helix of Col II. Recent evidence suggests that the cysteine protease cathepsin K may also cleave the triple helix of types I and II collagens but at different sites to MMPs.^{9,10}

Cathepsin K, a cysteine proteinase of the papain family, mediates the resorption of bone by osteoclasts^{13,27,28} and is considered a drug target for the treatment of osteoporosis in which bone resorption is excessive. This potentially very degradative enzyme is also expressed by other cells, including chondrocytes in articular cartilage.¹⁴

Cathepsin K is also capable of degrading other matrix molecules of hyaline cartilage, such as aggrecan and link protein.¹¹ The aggrecan-degrading activity of cathepsin K leads to the release of glycosaminoglycan-containing fragments that interact with the enzyme and form collagenolytically active complexes with increased stability at normal pH.¹⁰ Cathepsin K is the only cysteine protease that has also been shown to cleave telopeptide domains as well as α-chains of fibrillar collagens at multiple sites.¹² Degradation of telopeptides leads to depolymerization of the fibrillar network, whereas cleavage of the triple helix results in depolymerisation and denaturation.

Lafienah et al. discloses that cathepsin K is capable of cleaving in vitro the α-chains of purified native soluble type I and II collagen.⁹ Lafienah et al. identified one of several initial degradation products, which is obtained by cathepsin K cleavage close to the N terminus of the triple helix located 58 residues from the N terminus, at a Gly-Lys bond. However, it is unclear whether products with either of the new termini resulting from cleavage at this position are retained in the tissue rather than being degraded to freely diffusible non-antigenic peptides. Indeed, many studies (Li et al., 2004, J. Biol. Chem. 279, 5470-5479; Atley et al., 2000, Bone 26, 241-247; Li et al., 2000, Biochemistry 39, 529-536; Garnero et al., 1998, J. Biol. Chem. 273, 32347-32352) have suggested that cathepsin K generates a large scale degradation of the collagen substrate. It therefore remains unclear whether there are any in vivo collagen peptide generated by cathepsin K and in the event there are any, whether such peptides would be detectable.

Further, it is unclear how cathepsin K may interact in vivo with in vitro identified target site(s) within the intact triple helix and/or whether cathepsin K actually has any activity in vivo against type II collagen.⁹

Very little is known about the involvement of cathepsin K, if any, in the degradation of bone, cartilage and/or soft tissue collagen under physiological and pathological conditions. There is therefore a need for an assay for detecting cathepsin K activity in bone, cartilage and/or soft tissue.

### SUMMARY OF THE INVENTION

Accordingly, there is provided a method and kit for detecting cathepsin K activity in bone, cartilage and/or soft tissue.

In a broad aspect, there is provided a method for detecting cathepsin K activity in bone, cartilage and/or soft tissue, the method comprising providing a biological sample, contacting the sample with an antibody having binding specificity for a neoepitope comprising EAGKPG, and detecting an immunocomplex formed between the antibody and the neoepitope, wherein presence of the immunocomplex in the sample is indicative of cathepsin K activity in bone, cartilage and/or soft tissue.

In another broad aspect, there is provided a kit for detecting cathepsin K activity in bone, cartilage and/or soft tissue, the kit comprising an antibody having binding specificity for a neoepitope comprising EAGKPG, and instructions for detecting cathepsin K activity in a biological sample.

In one embodiment, the herein defined method and kit comprises an antibody having binding specificity for a neoepitope comprising GEAGKPG.

In one embodiment, the herein defined method comprises contacting the immunocomplex with a label reagent.

In one embodiment, the herein defined biological sample is a human sample.

In one embodiment, the herein defined biological sample is any biological fluid, cell sample, or tissue, as long as it contains, or is suspected of containing the herein defined neoepitope. In one embodiment, the herein defined biological sample is blood, urine or synovial fluid.

In one embodiment, the herein defined presence of cathepsin K activity is indicative of cartilage degeneration. In one embodiment, the herein defined cartilage degeneration is caused by osteoarthritis and/or age associated degeneration.

In one embodiment, the herein immunocomplex formed between the antibody and the herein defined neoepitope is detected by an assay selected from the group consisting of enzyme linked immunosorbent assay (ELISA), radioimmunoassay (RIA), immunofluorescent assay (IFA), and immunohistochemistry assay.

In one embodiment, the herein immunocomplex formed between the antibody and the herein defined neoepitope is indicative of type I and/or II collagen degradation by cathepsin K.

The following examples are presented in order to provide a more detailed description of specific embodiments of the represent invention and are not to be construed as limiting the scope of the invention.

### BRIEF DESCRIPTION OF THE DRAWINGS

In the figures, which illustrate, by way of example only, embodiments of the present invention:

FIG. 1. Immunoassay standard curve and epitope specificity of the antiserum R771. Percent inhibition obtained with serial dilutions of the immunizing and competing peptides with amino acid addition or deletion at the carboxy terminus (A) or modification of the amino terminus (B).

FIG. 2. Concentration-dependent generation of C2K neoepitope by cathepsin K. Human Col II (0.6 mg/ml) was digested for 3 hours with various concentrations of cathepsin K. Neoepitope was measured by competitive ELISA.

FIG. 3. Time courses of cleavage of triple helical human Col II by cathepsin K at pH 5.5 and pH 7.0. The generation of the C2K neoepitope was measured by competitive ELISA.

FIG. 4. Effect of chondroitin sulfate on cathepsin K activity with time. Col II was digested with cathepsin K in the absence and presence of either chondroitin 4-sulfate (C4S) or chondroitin 6-sulfate (C6S), at pH 7.0. The generation of the cathepsin K neoepitope was measured by competitive ELISA.

FIG. 5. Digestion of normal articular human cartilage with cathepsin K. The digestions were performed at pH 5.5 and the release of C2K neoepitope (A) and GAG (B) throughout time was measured by competitive ELISA and the DMMB assay, respectively.

FIG. 6. C2C and C2K neoepitope contents in normal and OA articular cartilages. The cartilage was digested with α-chymotrypsin, then either C2C (A) or C2K (B) neoepitope levels were quantitated in the digests by competitive ELISAs. Nonarthritic cartilages were divided into two groups: N1 (patient ages, 15 to 38 years) and N2 (patient ages, 41 to 70 years); patient ages for OA cartilages ranged between 49 to 87 years. In both cases the difference between the median (bar) of neoepitope content in the nonarthritic and the OA cartilages was determined by Mann-Whitney analysis and the P values are shown in the figures.

FIG. 7. Correlations between the content of C2C (MMP collagenase-generated neoepitope) and C2K neoepitopes in articular cartilages. Data from FIG. 6 were used to show the correlation between C2C and C2K neoepitope content in nonarthritic (A) and OA (B) cartilages. Statistically significant relationships between the two neoepitope contents were determined by Spearman rank correlation analysis (n, r, and P values are shown in the figure).

FIG. 8. Reduction in the generation of C2K neoepitope by a specific cathepsin K inhibitor. OA explants from eight patients were cultured in the presence and absence of L-873724 inhibitor for 16 days. The content of the C2K neoepitope at the end of the culture was determined by competitive ELISA. The age and gender of each patient is indicated. P values, determined by one-way analysis of variance test and less than 0.05, are considered significant compared to control and are indicated (asterisk).

FIGS. 9 A to D. Clustal Format™ sequence alignment between human alpha-1 type I collagen, human alpha-1 type II collagen and human alpha-2 type I collagen, showing in a box the presence of a sequence corresponding to the herein defined C2K neoepitope in both human alpha-1 type I collagen and human alpha-1 type II collagen.

### DETAILED DESCRIPTION OF THE INVENTION

The inventors have discovered that the degradation of collagen under physiological and/or pathological conditions by the proteolytic action of cathepsin K results in the generation of a neoepitope (C2K), which may therefore be used as a biomarker for cathepsin K activity.

To illustrate this concept, the inventors have made an antibody against the C-terminal neoepitope (C2K) generated in triple-helical type II collagen by the proteolytic action of cathepsin K.

In one embodiment, the anti-C2K antibody may be used in a method for detecting the presence of C2K neoepitope in a biological sample, which is indicative of cathepsin K activity in bone, cartilage and/or soft tissue under physiological and pathological conditions.

The herein described method is generally based on, but not limited to, an immunoassay, such as ELISA (enzyme-linked immunosorbent assay), for detection of the C2K neoepitope (hereinafter “antigen”).

The term “immunoassay” as used herein refers to an analytical method which uses the ability of an antibody to bind a particular antigen, thus forming an immunocomplex, as the means for determining the presence of the antigen. In general, the antibody is immobilized on a support and is capable of binding to the antigen in a biological sample. The antigen is provided by the biological sample. In a variation, the antibody is mixed with the antigen in the biological sample and the antigen-antibody complex thus formed (immunocomplex) is captured by a second antibody against the antigen or antibody or both in the antigen-antibody complex, where the second antibody is immobilized on a support. Alternatively, the formation of the antigen-antibody complex is measured in solution.

It is contemplated that a range of immunoassay formats be encompassed by this definition, including but not limited to direct immunoassays, indirect immunoassays, and “sandwich” immunoassays. One format is a sandwich enzyme-linked immunosorbent assay (ELISA). However, it is not intended that the present invention be limited to this format. It is contemplated that other formats, including radioimmunoassays (RIA), immunofluorescent assays (IFA), immunohistochemistry assay, and other assay formats, including, but not limited to, variations on the ELISA method will be useful in the method of the present invention. Thus, other antigen-antibody reaction formats may be used in the present invention, including but not limited to “flocculation” (i.e., a colloidal suspension produced upon the formation of antigen-antibody complexes), “agglutination” (i.e., clumping of cells or other substances upon exposure to antibody), “particle agglutination” (i.e., clumping of particles coated with antigen in the presence of antibody or the clumping of particles coated with antibody in the presence of antigen); “complement fixation” (i.e., the use of complement in an antibody-antigen reaction method), and other methods commonly used in serology, immunology, immunocytochemistry, histochemistry, and related fields.

Detection of an antibody-C2K neoepitope antigen immunocomplex can be performed by several methods. The antibody may be prepared with a label such as biotin, an enzyme, a fluorescent marker, or radioactivity, and may be detected directly using this label. Alternatively, a labeled “secondary antibody” or “reporter antibody” which recognizes the antigen and/or the first antibody may be added, forming a complex comprised of primary antibody-antigen-secondary antibody. Again, appropriate reporter reagents are then added to detect the labeled antibody. Any number of additional antibodies may be added as desired. These antibodies may also be labeled with a marker, including, but not limited to, an enzyme, fluorescent marker, or radioactivity. The antibody (primary or secondary) may be immobilized on a solid support, but the labeled component cannot be immobilized because the detectable signal is precluded from being a measure of binding.

As used herein, the term “reporter reagent” is used in reference to compounds which are capable of detecting the presence of antibody bound to antigen. For example, a reporter reagent may be a colorimetric substance which is attached to an enzymatic substrate. Upon binding of antibody and antigen, the enzyme acts on its substrate and causes the production of a color. Other reporter reagents include, but are not limited to, fluorogenic and radioactive compounds or molecules. This definition also encompasses the use of biotin and avidin-based compounds (e.g., including compounds, but not limited to, neutravidin and streptavidin) as part of the detection system. In one embodiment of the present invention, biotinylated antibodies may be used in the present invention in conjunction with avidin-coated solid support.

As used herein, the term “solid support” is used in reference to any solid material to which reagents such as antibodies, antigens, and other compounds may be attached. For example, in the ELISA method, the wells of microtiter plates often provide solid supports. Other examples of solid supports include microscope slides, coverslips, beads, particles, cell culture flasks, as well as many other items known to the person skilled in the art.

A kit for detecting cathepsin K activity in bone, cartilage and/or soft tissue generally comprises, in an amount sufficient for at least one assay, an anti-C2K neoepitope antibody, and optionally, means for detecting an immunocomplex between the C2K neoepitope antigen and the anti-C2K neoepitope antibody, as separately packaged immunochemical reagents. Instructions for use of the packaged immunochemical reagent are also typically included.

As used herein, the term “packaged” can refer to the use of a solid matrix or material such as glass, plastic, paper, fiber, foil and the like capable of holding within fixed limits an antibody of this invention. Thus, for example, a package can be a glass vial used to contain milligram quantities of a contemplated antigen or it can be a microtiter plate well to which microgram quantities of a contemplated antigen has been operatively affixed. Alternatively, a package could include anti-C2K neoepitope antibody-coated microparticles entrapped within a porous membrane or embedded in a test strip or dipstick, etc. Alternatively, the anti-C2K neoepitope antibody can be directly coated onto a membrane, test strip or dipstick, etc. which contacts the sample fluid. Many other possibilities exist and will be readily recognized by those skilled in this art.

Instructions for use typically include a tangible expression describing the reagent concentration or at least one assay method parameter such as the relative amounts of reagent and sample to be admixed, maintenance time periods for reagent/sample admixtures, temperature, buffer conditions and the like.

In one embodiment, a kit in accordance with the present invention further includes a label or indicating means capable of signaling the formation of a complex between the C2K neoepitope antigen and the anti-C2K neoepitope antibody.

As used herein, the terms “label” and means for detecting the antibody-C2K neoepitope antigen complex (“indicating means”) refer to molecules that are either directly or indirectly involved in the production of a detectable signal to indicate the presence of a complex. Any label or indicating means can be linked to or incorporated in an expressed protein, peptide, or antibody molecule that is part of the present invention, or used separately, and those atoms or molecules can be used alone or in conjunction with additional reagents. Such labels are themselves well known in clinical diagnostic chemistry.

The labeling means can be a fluorescent labeling agent that chemically binds to antibodies or antigens without denaturing them to form a fluorochrome (dye) that is a useful immunofluorescent tracer. Suitable fluorescent labeling agents are fluorochromes such as fluorescein isocyanate (FIC), fluorescein isothiocyante (FITC), 5-dimethylamine-1-natpthalenesulfonyl chloride (DANSC), tetramethylrhodamine isothiocyanate (TRITC), lissamine, rhodamine 8200 sulphonyl chloride (RB 200 SC) and the like.

In one embodiment, the indicating group is an enzyme, such as horseradish peroxidase (HRP), glucose oxidase, or the like. In such cases where the principle indicating group is an enzyme such as HRP or glucose oxidase, additional reagents are required to indicate that a receptor-ligand complex (immunoreactant) has formed. Such additional reagents for HRP include hydrogen peroxide and an oxidation dye precursor such as diaminobenzidine. An additional reagent useful with glucose oxidase is 2,2,-azino-di-(3-ethyl-benzthiazoline-G-sulfonic acid) (ABTS).

Radioactive elements are also useful labeling agents and are used illustratively herein. An exemplary radiolabeling agent is a radioactive element that produces gamma ray emissions. Elements which themselves emit gamma rays, such as ¹²⁴I, ¹²⁵I, ¹²⁸I, ¹³²I and ⁵¹ Cr represent one class of gamma ray emission-producing radioactive element indicating groups. Particularly preferred is ¹²⁵I. Another group of useful labeling means are those elements such as ¹¹C, ¹⁸F, ¹⁵O and ¹³N which themselves emit positrons. Also useful is a beta emitter, such as ¹¹¹indium or ³H.

The linking of labels, i.e., labeling of peptides and proteins is well known in the art. For instance, monoclonal antibodies produced by a hybridoma can be labeled by metabolic incorporation of radioisotope-containing amino acids provided as a component in the culture medium. The techniques of protein conjugation or coupling through activated functional groups are particularly applicable.

The methods and kits described herein can also include, optionally as a separate package, a “specific binding agent,” which is capable of selectively binding an antibody or antigen of this invention or a complex containing such a species, but is not itself antigen or antibody of this invention. Exemplary specific binding agents are second antibody molecules, e.g. complement proteins or fragments thereof, S. aureus protein A, and the like. Preferably the specific binding agent binds the antibody or antigen when it is present as part of a complex.

In one embodiment, the specific binding agent is labeled. However, when the herein described method or kit includes a specific binding agent that is not labeled, the agent is typically used as an amplifying means or reagent to amplify the signal. In these embodiments, the labeled specific binding agent is capable of specifically binding the amplifying means when the amplifying means is bound to a complex.

In one embodiment, the herein described kits can be used in an “ELISA” format to detect the quantity of C2K neoepitope antigen in a fluid sample or extract. “ELISA” refers to an enzyme linked immunosorbent assay such as those discussed above, which employ an antibody bound to a solid phase and an enzyme-antigen or enzyme-antibody conjugate to detect and quantify the amount of an antigen present in a sample.

In a number of embodiments, an anti-C2K neoepitope antibody can be affixed to a solid matrix to form a solid support. A reagent is typically affixed to a solid matrix by adsorption from an aqueous medium, although other modes of affixation applicable to proteins and peptides well known to those skilled in the art can be used.

Useful solid matrices are also well known in the art. Such materials are water insoluble and include the crosslinked dextran (e.g., Sephadex™ from Pharmacia Fine Chemicals, Piscataway, N.J.); agarose; polystyrene beads generally about 1 micron to about 5 millimeters in diameter polyvinyl chloride, polystyrene, crosslinked polyacrylamide, nitrocellulose- or nylon-based webs such as sheets, strips or paddles; or tubes, plates or the wells of a microtiter plate such as those made from polystyrene or polyvinylchloride.

The immunoreagents of any diagnostic system described herein can be provided in solution, as a liquid dispersion or as a substantially dry powder, e.g., in lyophilized form. Where the indicating means is an enzyme, the enzyme's substrate can also be provided in a separate package. A solid support such as the above-described microtiter plate and one or more buffers can also be included as separately packaged elements in the diagnostic assay systems of this invention.

In one embodiment, the anti-C2K neoepitope antibody is coated or adsorbed on to the surface of a substrate. A sample of interest is contacted with the anti-C2K neoepitope antibody and any C2K neoepitope antigen which may be present in the sample binds to the antibody. Secondary anti-C2K neoepitope antibodies, which may be the same or different antibody as the coated or adsorbed anti-C2K neoepitope antibody, are contacted with the C2K neoepitope antigen/sample and bind to the C2K neoepitope antigen that are bound to the anti-C2K neoepitope antibody.

The secondary anti-C2K neoepitope antibodies may be detected with a detectable label. The label is any entity that is capable of being conjugated or bound to the anti-C2K neoepitope antibody and that is capable of being detected by an analytical technique. The label may be conjugated or bound to the anti-C2K neoepitope antibody prior to or after contacting the anti-C2K neoepitope antibody with the C2K neoepitope antigen/sample.

Detection of the label is an indication that the C2K neoepitope antigen is present in the sample. If the label cannot be detected, then this is an indication that the antigen is not in the sample.

In one embodiment, the label may be a chemical moiety capable of being detected by an analytical technique, the chemical moiety being conjugated to the anti-human antibody. In one embodiment, the chemical moiety may be conjugated to the secondary anti-C2K neoepitope antibody before the secondary anti-C2K neoepitope antibody is contacted with the C2K neoepitope antigen/sample.

In another embodiment the label may be another antibody or collection of other antibodies having conjugated thereto a chemical moiety that is capable of being detected by an analytical technique. In one embodiment, the other antibody or collection of other antibodies having the chemical moiety conjugated thereto may be bound to the anti-C2K neoepitope antibody after the anti-C2K neoepitope antibody is contacted with the C2K neoepitope antigen/sample.

In one embodiment, the method further comprises a washing step before detecting the immunocomplex, and more preferably between each of the steps of the method. Washing is preferably accomplished using a washing solution comprising a buffer, such as phosphate buffered saline solution containing about 1% normal serum from the animal species in which the antibody to which the chemical moiety is conjugated was prepared. An emulsifier may also be present in the washing solution.

A variety of chemical moieties capable of being detected by an analytical technique and capable of being conjugated to an antibody may be used in the label. For instance, the chemical moiety may be capable of fluorescence or radioactivity (see Fuller S. A., Evelegh M. J. & Hurrell J. G. R. (2000) Conjugates of Enzymes to Antibodies. In: Current Protocols in Molecular Biology. (Eds. Ausubel F. M., Brent R., Kingston R. E., Moore D. D., Seidman J. G., Smith J. A. & Struhl K.) John Wiley & Sons, Inc. Vol. 2, pp. 11.1.1-11.1.7, and, Sambrook J., Fritsch E. F. & Maniatis T. (1989) Molecular cloning. A laboratory manual. 2^{nd} Ed. Cold Spring Harbor Laboratory Press. Cold Spring Harbor, N.Y., the disclosure of both being hereby incorporated by reference). An example of chemical moiety useful in this invention is alkaline phosphatase. Further treatment may be required before the analytical technique is used to detect the label depending on the particular technique or chemical moiety being used.

Analytical techniques useful as detection methods are generally known in the art. For example, colorimetric, electrophoretic and radio-labeling techniques may be used (see Sambrook J., Fritsch E. F. & Maniatis T. (1989) Molecular cloning. A laboratory manual. 2^{nd} Ed. Cold Spring Harbor Laboratory Press. Cold Spring Harbor, N.Y., the disclosure of which is hereby incorporated by reference).

Colorimetric techniques are generally preferred and may employ spectroscopic or visual verification of a color change indicating a positive or negative test result. One skilled in the art will appreciate that other techniques may be employed as detection methods in the present invention.

The herein described kit may further comprise means for detecting the label, or such means may be separate from the kit.

The herein described antibody may be a polyclonal or a monoclonal antibody. As used herein, the term “antibody” means an immunoglobulin molecule or a fragment of an immunoglobulin molecule having the ability to specifically bind to a particular antigen. Antibodies are well known to those of ordinary skill in the science of immunology. As used herein, the term “antibody” means not only intact antibody molecules but also fragments of antibody molecules retaining antigen binding ability. Such fragments are also well known in the art and are regularly employed both in vitro and in vivo. In particular, as used herein, the term “antibody” means not only intact immunoglobulin molecules but also antigen binding active fragments such as the well-known active fragments F(ab′)₂, Fab, Fv, and single domain antibody (Fd). Techniques for obtaining a single domain antibody with at least some of the binding specificity of the intact antibody from which they are derived are known in the art. For example, Ward, et al. Nature 341: 644-646 (1989), disclose a method of screening to identify an antibody heavy chain variable region (V_{H} single domain antibody) with sufficient affinity for its target epitope to bind thereto in isolated form.

Monoclonal-antibody-producing cells are prepared by selecting from immunized warm-blooded animals an individual animal, in which the antibody titer is raised, collecting the spleen or the lymph node 2 to 5 days after the last immunization, fusing the antibody-producing cells present in these organs with myeloma cells and selecting mAb producing hybridomas. The fusion is carried out according to the known method such as the method of Kohler et al. (Nature, 256, 495 (1975)). The myeloma cells include but not limited, for example, PAI, P3U1 (Health Science Research Resources Bank; HSRRB), Japan, Catalogue No. JCRB0113 and JCRB0708) and the like. The fusion promotion agents include polyethylene glycol (PEG) and Sendai virus (HVJ), but preferably PEG with a molecular weight 1000 to 6000. An efficient cell fusion may be achieved by adding the promotion agent at a concentration of about 10 to 80% and incubating at 20 to 40° C.

Selection of monoclonal antibody (mAb) may be carried out in accordance with the known method. Generally, it is carried out in a cell culture medium for animal cells added with HAT (hypoxanthine, aminopterin, thymidine). The media for selection and growth may include, for example, PRMI1640 medium containing 10 to 20% bovine fetal serum and the like. Cells are generally cultured under 5% CO₂ gas at a temperature 20 to 40° C. for 5 days to 3 weeks.

The culture supernatants are collected from wells in which hybridoma cells are cultured, and antibodies reacting with the antigen peptide may be selected by the ELISA method. First, the antigen peptide is immobilized onto 96-well plates and then blocked with calf serum. After reacting the supernatant of hybridomas with Mouse Immunoglobulins/HRP (Amersham-Pharmacia) at 37° C. for 1 hour, color is developed using Tetra Methyl Benzidine Microwell Peroxidase Substrate (TMB; Funakoshi) as a substrate. After terminating the reaction by acid, absorbance at 450/540 nm is measured. The antibodies with the absorbance of about 3 are selected, and cloning is carried out by the limited dilution method.

The target hybridoma cells thus obtained are cultured and the monoclonal antibody may be obtained from the culture medium. Alternatively, the hybridoma cells may be inoculated intraperitonealy into, for example, mouse (Balb/c) and the monoclonal antibody may be obtained from the ascites. Purification of the monoclonal antibody may be carried out in a similar manner as the conventional separation and purification of polyclonal Ab. Several methods for obtaining monoclonal antibodies are known in the art and are set forth, for instance, in U.S. Pat. Nos. 7,501,494, 7,498,129, 7,494,650, 7,494,647, 7,491,516, 7,488,477, 7,482,128 and 7,479,543, which are incorporated by reference herein in their entirety.

The foregoing is considered as illustrative only of the principles of the invention. Further, since numerous modifications and changes will readily occur to those skilled in the art, it is not desired to limit the invention to the exact examples and embodiments shown and described, and accordingly, all suitable modifications and equivalents may be resorted to, falling within the scope of the invention.

### Examples

### Materials and Methods

### Tissue

At total knee arthroplasty full-depth human articular femoral condylar cartilages were obtained from 27 patients with knee OA diagnosed in accordance with the criteria of the American College of Rheumatology.¹⁵ Nonosteoarthritic femoral condylar cartilages were collected at autopsy from 17 individuals of different ages (17 to 70 years).

### Peptide Synthesis

The peptide chosen for the preparation of the immunogen is comprised in the primary C-terminal neoepitope (C2K) generated in triple-helical human Col II by the proteolytic action of cathepsin K (Table I).⁹ For the determination of the specificity of the anti-neoepitope antiserum, six other peptides were synthesized: a one-residue truncation (Table I, i) and a one-residue extension (Table I, ii) of the immunizing peptide at the C terminus and a sequence similar to that of the immunizing peptide, except for the proline residue that was 4-hydroxylated (Table I, iii). In addition, three other peptides were prepared to investigate the importance of the N-terminal portion of the epitope. These included deletion of either the G (Table I, iv) or A (Table I, v) or inversion of the AE dipeptide (Table I, vi). A cysteine residue was added to the common N terminus of each peptide to allow conjugation of the sulfhydryl group to the carrier proteins, ovalbumin and keyhole limpet hemocyanin. The peptides were synthesized at the Sheldon Biotechnology Centre, McGill University, or by CanPeptide Inc. (Pointe-Claire, Canada).

TABLE I
Sequences of the collagen α1 (II) chain in the region of the cathepsin K
cleavage site and the synthetic peptides used in the antisera preparation and
characterization.

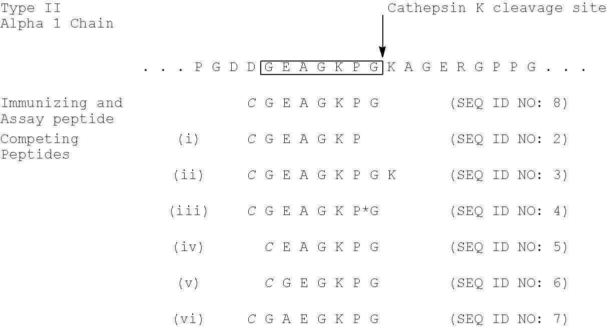

A cysteine residue (italics) was added to the N terminus for coupling to the carrier protein.
P* denotes 4-hydroxyproline.

### Production of Polyclonal Antibodies

The synthetic peptide comprised within the C2K neoepitope was coupled to keyhole limpet hemocyanin and the protein content was determined as previously described.¹⁶ The conjugate was used to immunize four young, disease-free, New Zealand White rabbits (female, 2.5 to 3.0 kg) (Veterinary School, University of Montreal, Step-Hyacinthe, Canada). Preimmune bleeds were obtained before immunization for use as control serum. Each rabbit was immunized on day 0 intramuscularly with a total of 0.5 mg of antigen emulsified with complete Freund's adjuvant. On days 14 and 28 the rabbits were boosted with similar quantities of keyhole limpet hemocyanin-conjugated peptide in Freund's incomplete adjuvant. On day 38 the rabbits were exsanguinated by cardiac puncture and serum was collected.

### Characterization of Antisera Titer and Specificity

A direct enzyme-linked immunosorbent assay (ELISA) assay was used to determine the antibody titer. The antiserum with the highest titer was selected for use. Investigation of the neoepitope specificity of the antiserum was performed by competitive inhibition ELISA as described.¹⁷ The percentage binding and percentage inhibition were calculated for each of the standard peptides and results were expressed on a molar basis.

### Inhibition ELISA for Analyses of Col II—Neoepitope Produced by Cathepsin K Cleavage

Immulon™ 2 plates (VWR International, Montreal, Canada) were coated with the peptide-ovalbumin conjugate (prepared as described above for the hemocyanin conjugate) diluted in phosphate-buffered saline (PBS), pH 7.2, at 3 ng/well (50 μl/well), the optimum concentration as determined by checkerboard analyses. After overnight incubation at 4° C., the plate was washed three times with PBS-0.1% Tween™ 20 and blocked with 100 μl of PBS-1% bovine serum albumin (BSA), at room temperature for 30 minutes. Serial dilutions of the standard peptide containing the epitope that had been used for immunization were prepared and added at 50 μl/well in triplicate to round bottom polypropylene plates used as preincubation plates; the antiserum was diluted 1:12,000 (optimum dilution as determined by checkerboard analyses) in PBS-1% BSA, 0.1% Tween 20, pH 7.2, and added to the plates (50 μl/well). In the nonspecific binding wells 50 μl of PBS-1% BSA and 50 μl of PBS-1% BSA, 0.1% Tween 20 were added; the maximum binding wells contained 50 μl of the diluted antiserum and 50 μl of PBS-1% BSA. After 1 hour incubation at 37° C., 50 μl of each preincubated well was transferred to the Immulon 2 flat-bottom, 96-well microtiter plates. The plates were incubated at 4° C. for 30 minutes and then washed three times with PBS-0.1% Tween 20. Alkaline phosphatase-conjugated goat antirabbit IgG (Sigma, St. Louis, Mo.), diluted 1:1000 in PBS-1% BSA, 0.1% Tween 20 was then added to each well (50 μl/well). The plate was incubated for 1 hour at 37° C., washed three times with PBS-0.1% Tween 20, and once with distilled water. One 5 mg tablet of alkaline phosphatase substrate (Sigma) was dissolved in 10 ml of 8.9 mmol/L diethanolamine buffer, pH 9.8, and added to the plate (50 μl/well). The optical density (405 nm) was read after 1 hour incubation. The detection limit of the newly developed assay was 2.8 ng/ml and the coefficients of variation (% CV) of the intra-assays and inter-assays were <10%. The analytic recovery was 87.2 to 97.3% and the recovery rates for the serial dilution were 92 to 108%.

Digestion of Human Col II with Cathepsin K, MMP-13, and MMP-3

Recombinant human procathepsin K was expressed in the Pichia pastoris, processed to the mature form and purified as described previously.¹⁸ Col II was dissolved in 0.2 mol/L acetic acid (2 mg/ml) and dialyzed at 4° C. against 50 mmol/L sodium acetate buffer, pH 5.5, containing 2 mmol/L dithiothreitol, 2 mmol/L ethylenediaminetetraacetic acid (EDTA), and 350 mmol/L NaCl.⁹ The digestion buffer had the same composition as the dialyzing buffer with the addition of 0.15% (w/v) chondroitin-4 sulfate (chondroitin sulfate A, Sigma). To observe the effect of increasing the enzyme concentration on the reaction rate, the first digestion was performed by using four different concentrations of cathepsin K (1, 3, 10, and 30 μmol/L) throughout a single digestion time course of 3 hours at room temperature (25° C.). The reaction was stopped by the addition of a specific cysteine protease inhibitor, E-64 (Peptide International, Louisville, Ky.), at a final concentration of 50 μmol/L. Two other digestions were performed at pH 5.5, in the absence and presence of a potent and selective nonbasic cathepsin K inhibitor,¹⁹ L-873724 (Merck Frosst, Kirkland, Canada). Digestions at pH 7.0 in the presence of 2 μmol/L cathepsin K were used to determine the time-dependent release of the epitope at 0, 3, 24, and 48 hours, in the absence or presence of chondroitin-4 sulfate or chondroitin-6 sulfate (chondroitin sulfate C, Sigma). The generation of the neoepitope was assessed by using the inhibition ELISA described above. The digestion of human Col II with MMP-13² was performed to determine whether that MMP-13, thought to be the dominant collagenase involved in Col II degradation in OA cartilage, can generate the neoepitope produced by cathepsin K. The ELISA assay was used to measure the generation of C2K neoepitope. The C2C assay²⁰ was used to demonstrate the collagenase activity of MMP-13. In addition, heat denatured Col II was digested overnight with 4-aminophenylmercuric acetate-activated MMP-3 (Triple Point Biologics, Forest Grove, Oreg.).²¹ The activity of the enzyme was demonstrated by sodium dodecyl sulfate-polyacrylamide gel electrophoresis (SDS-PAGE) and the ELISA assay was used to assess possible generation of C2K neoepitope.

Digestion of Normal Articular Cartilage with Cathepsin K

The generation and release of C2K neoepitope from the tissue was first assessed in vitro by incubating normal articular cartilage with cathepsin K. Thus, 60 mg of cartilage was sliced into cubes of 2×2 mm² and incubated in 2 ml of digestion buffer described above, without the addition of chondroitin sulfate (pH 7), containing 2 μmol/L of cathepsin K. The same conditions were used for the control, except that no enzyme was added. The incubation was performed at 25° C. with shaking and buffer aliquots were removed at 0, 0.5, 1, 2, 3, and 6 hours. Degradation of Col II and proteoglycan by cathepsin K was assessed by inhibition ELISA of C2K neoepitope and a modified colorimetric 1,9-di-methylmethylene blue (DMMB) dye-binding assay to detect glycosaminoglycan (GAG) release, respectively.²² Matrix molecule release into the buffer was expressed per mg weight of cartilage as pmol C2K and μg GAG.

Collagen Extraction from Articular Cartilage

The content of C2K and C2C neoepitopes in cartilage were determined by extraction with α-chymotrypsin (Sigma). This degrades denatured but not intact triple helical collagen, to solubilize the collagen neoepitopes that can then be assayed. Femoral condylar cartilages from 17 normal and 19 OA individuals were washed and digested with α-chymotrypsin, as previously described.²³ The digest was removed and stored at −20° C. until analyzed. To ensure that the cathepsin K-generated neoepitope is not cleaved by α-chymotrypsin, a separate digestion was first performed by treating a 50 ng/ml immunizing peptide solution with α-chymotrypsin as described above. No loss of epitope was observed. The concentration of the neoepitope released from the cartilage in the α-chymotrypsin digests was expressed as pmol of neoepitope/mg wet wt of cartilage (based on the molecular weights of the two neoepitope sequences used as standards in the ELISA assays: 717.8 Da for the C2K neoepitope and 972 Da for C2C).

### Explant Culture of OA Cartilage in the Presence and Absence of the Cathepsin K Inhibitor L-873724

Femoral condylar cartilages from eight OA patients were prepared and cultured as previously described.²⁴ Under these culture conditions explant viability was always preserved for the duration of the culture as revealed by progressive incorporation of radiolabeled amino acids over and above frozen and thawed controls (data not shown). Wet weights of 70 to 80 mg were placed in each of the wells of the culture plates (24-well Costar™ 3548 plate; Corning Inc., Corning, N.Y.). After 48 hours of preculture at 37° C. the media were changed (day 0) and then replaced every 4 days for a total of 16 days. The cultures were established in quadruplicate with and without the cathepsin K inhibitor (L-873724)¹⁹ at 10 nmol/L, 25 nmol/L, and 50 nmol/L in a final concentration of 0.1% dimethyl sulfoxide (DMSO). The inhibitor was added fresh to the medium at each change. The media collected from day 4 to day 16 and the recovered cartilage were stored at −20° C. until analysis after digestion with α-chymotrypsin. Complete digestion of the cartilage was ensured by further adding 1 ml/tube of 1 mg/ml proteinase K (Sigma) in 50 mmol/L Tris-HCl containing the proteinase inhibitors.² After overnight incubation at 56° C. no residue remained and the enzyme was inactivated by boiling the samples for 10 minutes. The proteinase K digests were used only for determination of residual cartilage GAG content. Separate studies revealed that proteinase K degrades the C2K neoepitope. The media and α-chymotrypsin digests were assayed by ELISA for the C2K neoepitope. The concentration of the neoepitope released from the cartilage was calculated by summation of the concentrations found both in the media and α-chymotrypsin digests and expressed as pmol of C2K/mg wet wt of cartilage as above. The media, a chymotrypsin and proteinase K digests were assayed by the DMMB assay as described above. GAG release into the media was expressed as a percentage of the total GAG that represents the summation of the GAG concentration present in the media and the one from the two cartilage extracts.

### Toxicity Analyses

The effect of L-873724 on the synthesis of Col II and other matrix proteins was assessed by measuring the incorporation of [³H] proline in cartilage culture. The tissue was maintained for 7 days as described above, in the presence and absence of the inhibitor at 10 nmol/L, 25 nmol/L, and 50 nmol/L and media were changed at days 4 and 7. On day 7, medium was supplemented with 25 μCi/ml of tritiated proline (Amersham, Buckinghamshire, UK) and culture continued for 48 hours. After culture, the cartilage was harvested and washed three times for 10 to 15 minutes in PBS to remove free label. The cartilage was digested with proteinase K as described above and radioactivity determined by scintillation counting.

### Statistical Analysis

Spearman rank correlations were used for analyzing the relationship between the amount of neoepitopes generated in articular cartilage by collagenases and cathepsin K. Mann-Whitney U-tests were used to compare the nonarthritic and OA groups, in terms of neoepitope contents detected by immunoassay. The difference between the median of neoepitope concentration in treated and nontreated samples in the inhibitor study was also determined by Mann-Whitney analysis.

### Results

### Direct-Binding ELISA for Determination of Antibody Titers

One primary and two booster immunizations with the peptide conjugate yielded specific antisera with titers from 1:8×10⁵ to 1:1×10⁶ in each of four rabbits, as detected by direct ELISA. A single serum of the highest titer (R771) was selected is for further study. The optimum dilution of the antiserum to be used in the immunoassay, determined by checkerboard analysis was 1:12,000.²

### Inhibition ELISA for Neoepitope Characterization

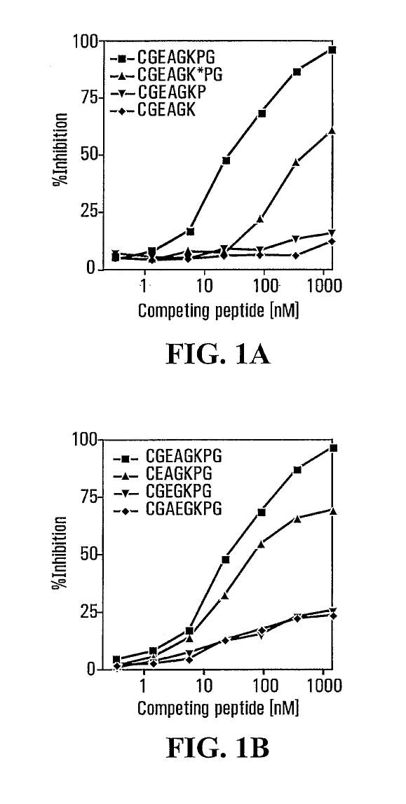

A standard inhibition curve was created by using a range of concentrations of the nonconjugated immunizing peptide. Six other inhibition curves were created using the other synthetic peptides (Table I) to assess specificity (FIGS. 1 A and B). The antibody has high affinity for the immunizing peptide (SEQ ID NO: 8) used alone as a competitive antigen in the ELISA inhibition assay, giving a median inhibitory concentration (IC50) of 45 nmol/L. The competition by, and thus binding of the antibody to, the competing synthetic peptides (i) and (ii) (truncated/extended forms of the immunizing peptides) (SEQ ID NO: 2 and 3, respectively) was negligible showing that removal or addition of residues at the C terminus resulted in a loss of inhibition (less binding to such peptides). The antibody showed reduced affinity for the form of the peptide containing hydroxyproline (peptide iii, FIG. 1A) (SEQ ID NO: 4). Although the antibody has some affinity for the N-terminally truncated peptide (iv) (SEQ ID NO: 5), negligible binding to competing peptides containing deletion of the alanine residue (v) (SEQ ID NO: 6) or where the glutamic acid and alanine residues were inverted (vi) (SEQ ID NO: 7) demonstrated the importance of the N-terminal region segment of the neoepitope (FIG. 1B).

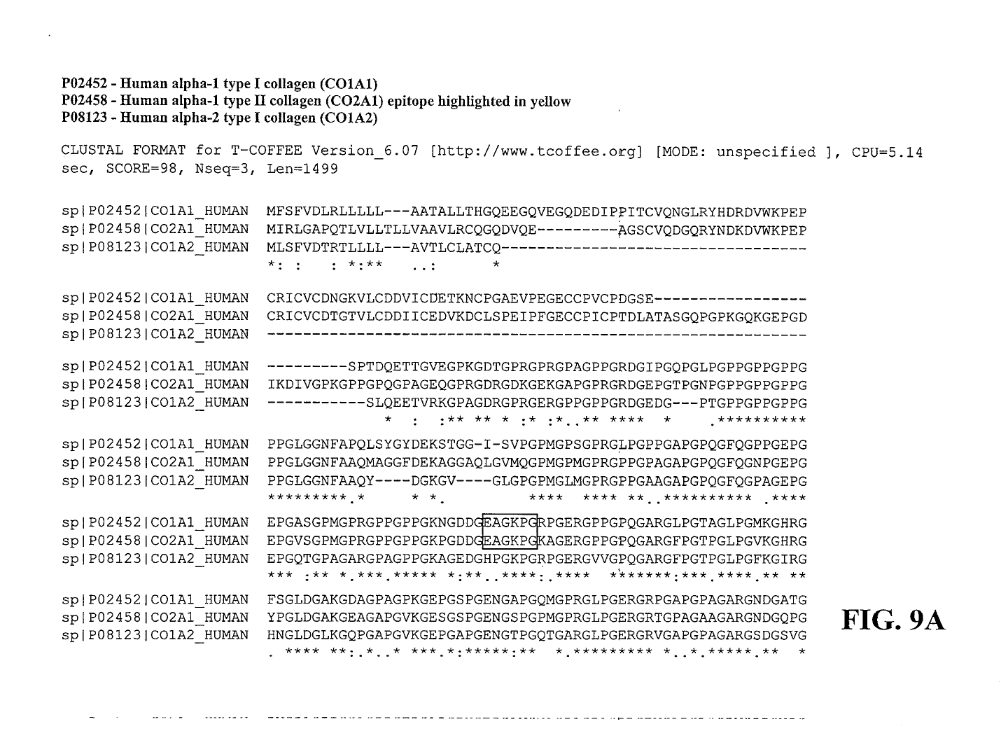

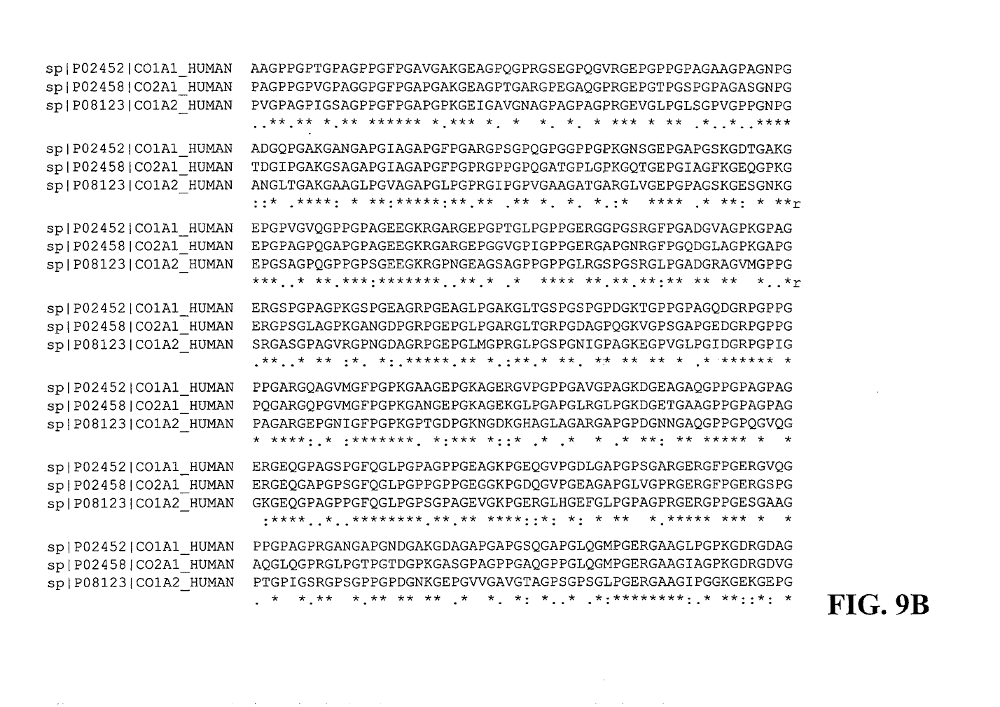

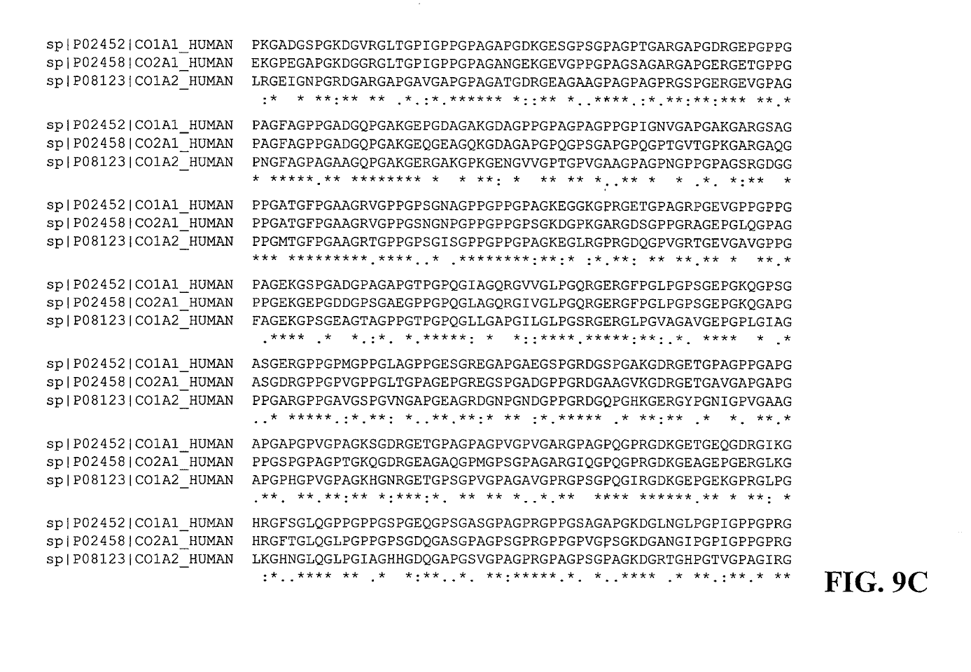

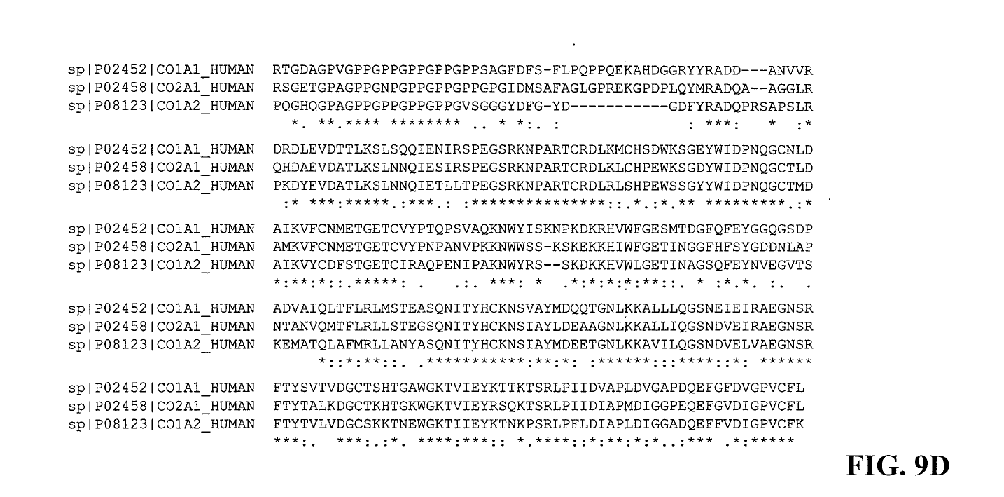

With these findings in mind it is important to point out that the neoepitope sequence EAGKPG (SEQ ID NO: 9) is unique for Col I and Col II based on a BLAST search²⁵ and the sequence alignment shown in FIG. 9. It is thus reasonable to predict that the herein described method may be used for detecting cathepsin K activity via detection of Col I and/or Col II C2K-containing degradation product in a biological sample.

Digestion of Human Col II with Cathepsin K, MMP-13, and MMP-3

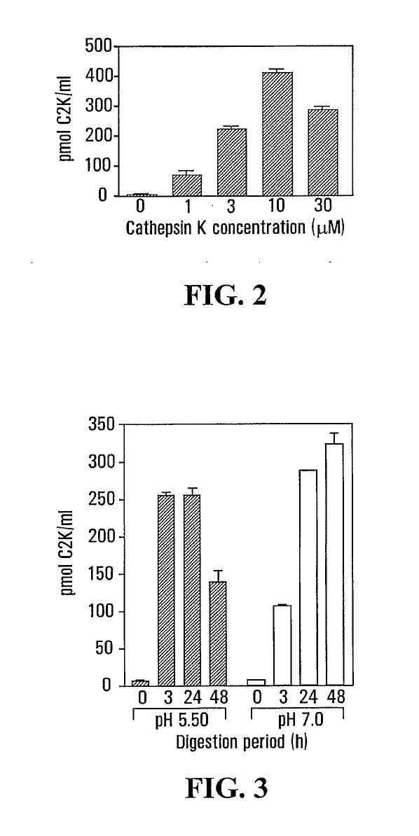

To further validate the specificity of the antibody to the neoepitope, Col II digested with increasing concentrations of cathepsin K was analyzed after 3 hours of incubation by competitive inhibition ELISA (FIG. 2). Although there was negligible reactivity with uncleaved Col II, with increasing concentrations of cathepsin K, an increase in the generation of neoepitope was detected, reaching a peak at 10 μmol/L followed by a decrease at 30 μmol/L. This suggests that higher concentrations of enzyme degrade the neoepitope because of gelatinolytic activity. To investigate the effect of pH, the time-dependent release of the neoepitope in the presence of a constant amount of enzyme at pH 5.5 and 7.0 was assessed.

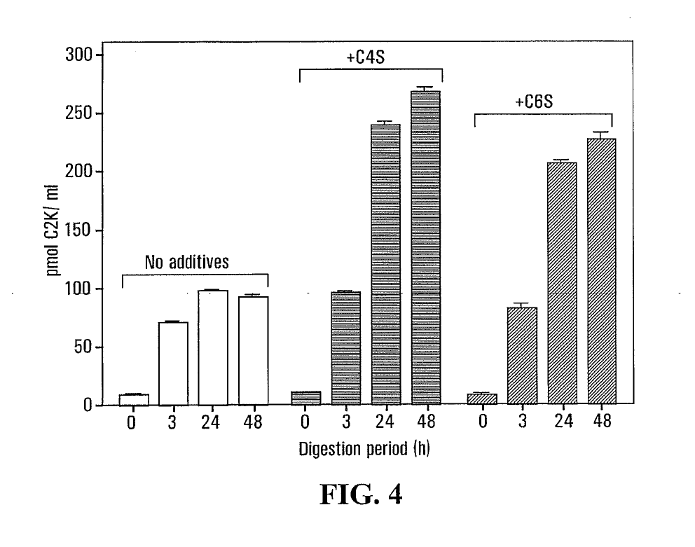

At pH 5.5, considered the optimum pH of cathepsin K,¹³ a rapid increase in the release of the neoepitope with a peak at 3 hours and a significant drop at 48 hours was observed, again suggesting that the neoepitope can be further degraded by the enzyme. When the specific cathepsin K inhibitor (L-873724) was added to the digestion buffer at the start of the incubation, the release of the neoepitope at each of the subsequent time points was negligible (data not shown). At pH 7.0 the release of the neoepitope was much slower and only reached a maximum at 24 to 48 hours (FIG. 3). When digestion was performed in the absence of chondroitin sulfate (CS) at pH 7.0 a decreased release of neoepitope was detected. The addition of C4S or C6S to the digestion buffer significantly increased the amount of C2K neoepitope, reaching a peak at 48 hours (FIG. 4). These findings suggest that chondroitin sulfate increases the activity and stability of the enzyme, as previously described.¹⁰ Cathepsin K did not generate the C2C neoepitope, as shown by analysis of collagen digests with C2C assay (data not shown). Moreover, the C2K neoepitope was not detected when collagen was digested by MMP-13 although digestion was confirmed by the C2C collagenase assay (data not shown). A review of peptide sequences known to be degraded by MMP-3²⁶ suggests the possibility that the C2K epitope could be generated by this protease after digestion of denatured Col II. However, when heat-denatured Col II was degraded with MMP-3 (full digestion being demonstrated by sodium dodecyl sulphate-polyacrylamide gel electrophoresis analysis) no C2K neoepitope was detected.

### Cathepsin K Can Digest Cartilage in Situ

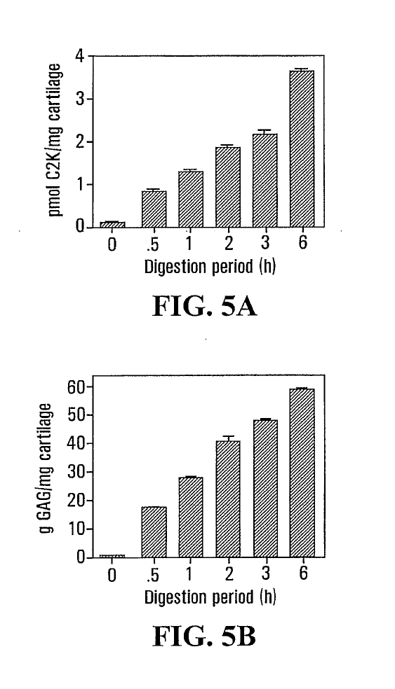

When cartilage was incubated with cathepsin K for 6 hours at pH 5.5, increasing amounts of the C2K neoepitope and GAG were detected in the supernatant. The release of GAG was detected from time 0 showing that cathepsin K efficiently degrades proteoglycans from the moment it is added to the cartilage (FIG. 5); time 0 was recorded after the enzyme was added to the digestion buffer containing the cartilage sample.

### Total Contents of Cathepsin K and Collagenase-Generated Neoepitopes in Human Articular Cartilages

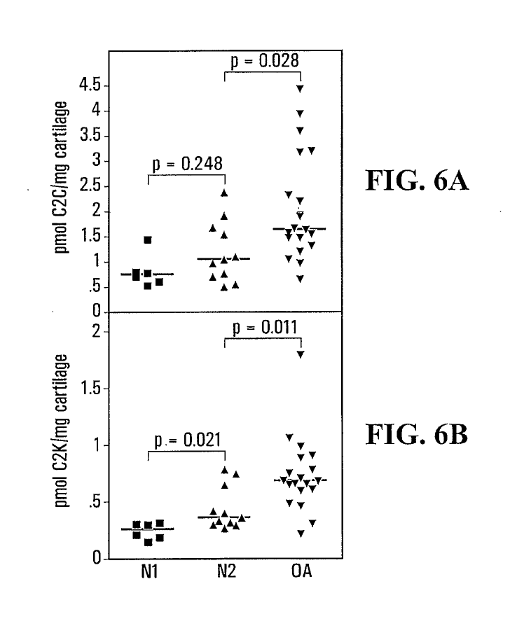

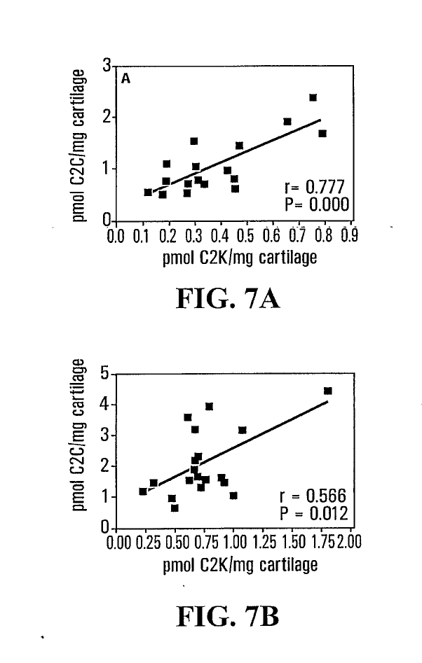

α-chymotrypsin extracts of denatured Col II from uncultured nonarthritic and OA cartilages were assayed for both cathepsin K-generated (C2K) and collagenase-generated (C2C) neoepitope contents. These were both increased in content with aging but only significantly in the case of the cathepsin K neoepitope (FIG. 6). In OA a significant increase was observed for both neoepitopes compared to adult articular cartilage. Strong positive and significant correlations were noted between cathepsin K and collagenase-generated neoepitope contents in nonarthritic cartilages (r=0.7765, P=0.0002) and in OA cartilages (r=0.5656, P=0.0115) (FIG. 7). The concentrations of the C2C neoepitope were usually two to three times higher than those of C2K.

### OA Articular Cartilage Explants Cultured With and Without Cathepsin K Inhibitor L-873724

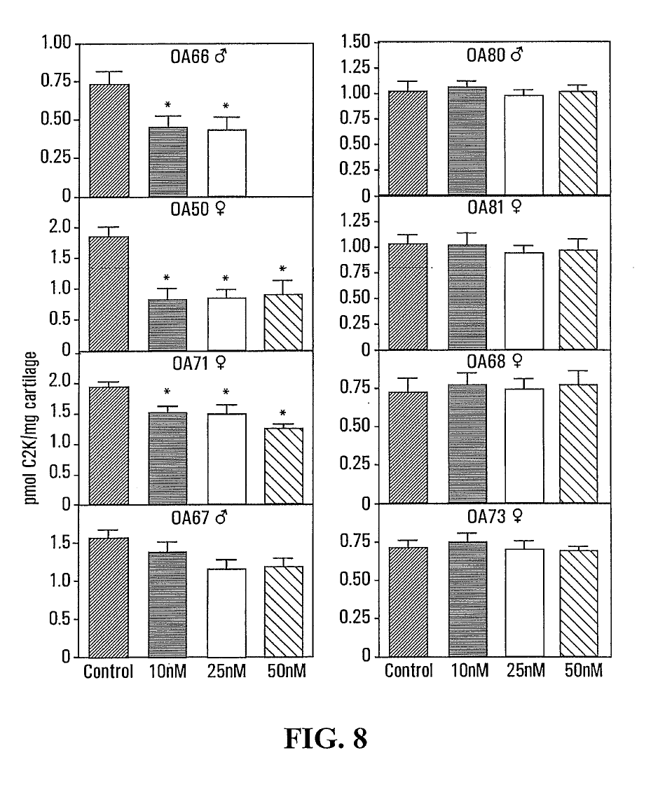

L-873724 is a selective inhibitor of human cathepsin K exhibiting an IC50 of 0.2 nmol/L against human cathepsin K and an 890-fold higher IC₅₀ value versus cathepsin B, L, and S. The inhibitor was used as a specific means of determining the involvement of this enzyme in the generation of the C2K neoepitope. The inhibitor was found to be nontoxic in that it did not affect the incorporation of tritiated proline into newly synthesized proteins at a range of concentrations including those used in our studies (data not shown). The C2K neoepitope was hardly detectable in some of these cultures in the culture media, being present mainly in cartilage. When these were totalled it was observed that the inhibitor reduced the generation of the neoepitope in four of eight OA patients with a statistically significant reduction being noted in three cases (FIG. 8). In one case study (OA66) there was insufficient cartilage to use the inhibitor at 50 nmol/L. The inhibitor had no effect on proteoglycan degradation measured as glycosaminoglycan release (data not shown).

These experimental data suggest that cathepsin K is involved in the cleavage of collagen in human articular cartilage in certain OA patients and that it may play a role in either or both OA pathophysiology and the aging process.

The person skilled in the art will appreciate that within the context of the herein described invention, levels of cathepsin K collagen degradation products in a biological sample, such as, but not limited to, blood (or urine or synovial fluid), may be used as measure (biomarker) of disease and/or disease progress. The antibodies herein described may also be used in species other than human (Vinardell et al., Osteoarthritis Cartilage, 2009 March; 17(3):375-83), and accordingly the herein described method and kits may be used for detecting cathepsin K activity in bone, cartilage and/or soft tissue of species other than human.

Although the present invention has been described in considerable detail with reference to certain embodiments thereof, variations and refinements are possible without departing from the spirit of the invention. Therefore, the scope of the invention should be limited only by the appended claims and their equivalents.

All references cited throughout the specification infra and supra, are hereby incorporated by reference in their entirety.

### LIST OF REFERENCES

1. Dodge G R, Poole A R: Immunohistochemical detection and immunochemical analysis of type II collagen degradation in human normal, rheumatoid and osteoarthritic articular cartilages and in explants of bovine articular cartilage cultured with interleukin 1. J Clin Invest 1989, 83:647-661.
2. Billinghurst R C, Dahlberg L, Ionescu M, Reiner A, Bourne R, Rorabeck C, Mitchell P, Hambor J, Diekmann O, Tschesche H, Chen J, Van Wart H, Poole A R: Enhanced cleavage of type II collagen by collagenases in osteoarthritic articular cartilage. J Clin Invest 1997, 99:1534-1545.
3. Mitchell P G, Magna H A, Reeves L M, Lopresti-Morrow L L, Yocum S A, Rosner P J, Geoghegan K F, Hambor J E: Cloning, expression, and type II collagenolytic activity of matrix metalloproteinase-13 from human osteoarthritic cartilage. J Clin Invest 1996, 97:761-768.
4. Dahlberg L, Billinghurst R C, Manner P, Nelson F, Webb G, Ionescu M, Reiner A, Tanzer M, Zukor D, Chen J, Van Wart H E, Poole A R: Selective enhancement of collagenase-mediated cleavage of resident type II collagen in cultured osteoarthritic cartilage and arrest with a synthetic inhibitor that spares collagenase 1 (matrix metalloproteinase). Arthritis Rheum 2000, 43:673-682.
5. Hollander A P, Heathfield T F, Webber C, Iwata Y, Bourne R, Rorabeck C, Poole A R: Increased damage to type II collagen in osteoarthritic articular cartilage detected by a new immunoassay. J Clin Invest 1994, 93:1722-1732.
6. Hollander A P, Pidoux I, Reiner A, Rorabeck C, Bourne R, Poole A R: Damage to type II collagen in aging and osteoarthritis starts at the articular surface, originates around chondrocytes, and extends into the cartilage with progressive degeneration. J Clin Invest 1995, 96:2859-2869.
7. Kempson G E: Relationship between the tensile properties of articular cartilage from the human knee and age. Ann Rheum Dis 1982, 41:508-511.
8. Miller E J, Harris E D Jr, Chung E, Finch J E Jr, McCroskery P A, Butler W T: Cleavage of type II and III collagens with mammalian collagenase: site of cleavage and primary structure at the NH2-terminal portion of the smaller fragment released from both collagens. Biochemistry 1976, 15:787-792.
9. Kafienah W, Brömme D, Buttle D J, Croucher L J, Hollander A P: Human cathepsin K cleaves native type I and II collagens at the Nterminal end of the triple helix. Biochem J 1998, 331:727-732.
10. Li Z, Hou W S, Brömme D: Collagenolytic activity of cathepsin K is specifically modulated by cartilage-resident chondroitin sulfates. Biochemistry 2000, 39:529-536.
11. Hou W S, Li Z, Buttner F H, Bartnik E, Bromme D: Cleavage site specificity of cathepsin K toward cartilage proteoglycans and protease complex formation. Biol Chem 2003, 384:891-897.
12. Garnero P, Borel O, Byrjalsen I, Ferreras M, Drake F H, McQueney M S, Foged N T, Delmas P D, Delaisse J M: The collagenolytic activity of cathepsin K is unique among mammalian proteinases. J Biol Chem 1998, 273:32347-32352.
13. Brömme D, Okamoto K, Wang B B, Biroc S: Human cathepsin O2, a matrix protein-degrading cysteine protease expressed in osteoclasts. Functional expression of human cathepsin O2 in Spodoptera frugiperda and characterization of the enzyme. Biol Chem 1996, 271:2126-2132.
14. Konttinen Y T, Mandelin J, Li T F, Salo J, Lassus J, Liljestrom M, Hukkanen M, Takagi M, Virtanen I, Santavirta S: Acidic cysteine endoproteinase cathepsin K in the degeneration of the superficial articular hyaline cartilage in osteoarthritis. Arthritis Rheum 2002, 46:953-960.
15. Altman R, Asch E, Bloch D, Bole G, Borenstein D, Brandt K, Christy W, Cooke T D, Greenwald R, Hochberg M: Development of criteria for the classification and reporting of osteoarthritis. Classification of osteoarthritis of the knee. Diagnostic and Therapeutic Criteria Committee of the American Rheumatism Association. Arthritis Rheum 1986, 29:1039-1049.
16. Billinghurst R C, Ionescu M, Poole A R: Immunoassay for collagenase mediated cleavage of types I and II collagens. Methods Mol Biol 2001, 151:457-472.
17. Mort J S, Roughley P J: Production of antibodies against degradative neoepitopes in aggrecan. Methods in Molecular Medicine, vol 100: Cartilage and Osteoarthritis, vol. 1: Cellular and Molecular Tools. Edited by Sabatini M, Pastoureau P, De Ceuninck F. Totowa, Humana Press, 2004, pp 237-249.
18. Billington C J, Mason P, Magny M-C, Mort J S: The slow-binding inhibition of cathepsin K by its propeptide. Biochem Biophys Res Commun 2000, 276:924-929.
19. Li C S, Deschenes D, Desmarais S, Falgueyret J P, Gauthier J Y, Kimmel D, Léger S, Masse F, McGrath M E, McKay D J, Percival M D, Riendeau D, Rodan S B, Thérien M, Truong V-L, Wesolowski G, Zamboni R, Black W C: Identification of a potent and selective non-basic cathepsin K inhibitor. Bioorg Med Chem Lett 2006, 16:1985-1989.
20. Poole A R, Ionescu M, Fitzcharles M A, Billinghurst R C: The assessment of cartilage degradation in vivo: development of an immunoassay for the measurement in body fluids of type II collagen cleaved by collagenases. J Immunol Methods 2004, 294:145-153.
21. Gunja-Smith Z, Woessner J F Jr: Activation of cartilage stromelysin-1 at acid pH and its relation to enzyme pH optimum and osteoarthritis. Agents Actions 1993, 40:228-231
22. Farndale R W, Buttle D J, Barrett A J: Improved quantitation and discrimination of sulphated glycosaminoglycans by use of dimethylmethylene blue. Biochim Biophys Acta 1986, 833:173-177.
23. Billinghurst R C, Wu W, Ionescu M, Reiner A, Dahlberg L, Chen J, Van Wart H, Poole A R: Comparison of the degradation of type II collagen and proteoglycan in nasal and articular cartilages induced by interleukin-1 and the selective inhibition of type II collagen cleavage by collagenase. Arthritis Rheum 2000, 43:664-672.
24. Kobayashi M, Squires G R, Mousa A, Tanzer M, Zukor D J, Antoniou J, Feige U, Poole A R: Role of interleukin-1 and tumor necrosis factor alpha in matrix degradation of human osteoarthritic cartilage. Arthritis Rheum 2005, 52:128-135.
25. Altschul S F, Gish W, Miller W, Myers E W, Lipman D J: Basic local alignment search tool. J Mol Biol 1990, 215:403-410.
26. Nagase H, Fields G B: Human matrix metalloproteinase specificity studies using collagen sequence-based synthetic peptides. Biopolymers 1996, 40:399-416.
27. Shi G-P, Chapman H A, Bhairi S M, DeLeeuw C, Reddy V Y, Weiss S J: Molecular cloning of human cathepsin O, a novel endoproteinase and homologue of rabbit OC2. FEBS Lett 1995, 357:129-134.
28. Brömme D, Okamoto K: Human cathepsin O2, a novel cysteine protease highly expressed in osteoclastomas and ovary. Molecular cloning, sequencing and tissue distribution. Biol Chem Hoppe-Seyler 1995, 376:379-384.
29. Li Z, Hou W S, Escalante-Torres C R, Gelb B D, Brömme D: Collagenase activity of cathepsin K depends on complex formation with chondroitin sulfate. J Biol Chem 2002, 277:28669-28676.
30. Rawlings N D, Morton F R, Barrett A J: MEROPS: the peptidase database. Nucleic Acids Res 2006, 34(Suppl 1):D270-D272

We claim:

1. A method for detecting cathepsin K activity in bone, cartilage and/or connective tissue, said method comprising providing a biological sample, contacting said sample with an antibody having binding specificity for a neoepitope comprising EAGKPG (SEQ ID NO: 9), and detecting an immunocomplex formed between said antibody and said neoepitope, wherein presence of said immunocomplex is indicative of cathepsin K activity in bone, cartilage and/or connective tissue.

2. The method of claim 1, wherein said neoepitope comprises GEAGKPG (SEQ ID NO: 1).

3. The method of claim 1, wherein said detecting said immunocomplex formed between said antibody and said neoepitope comprises contacting said immunocomplex with a detectable label.

4. The method of claim 1, wherein said cartilage is human cartilage.

5. The method of claim 1, wherein presence of said immunocomplex is indicative of cathepsin K activity in cartilage, and said cathepsin K activity in cartilage is indicative of cartilage degeneration.

6. The method of claim 5, wherein said cartilage degeneration is caused by osteoarthritis and/or age associated degeneration.

7. The method of claim 1, wherein said detecting said immunocomplex formed between said antibody and said neoepitope is by an assay selected from the group consisting of enzyme linked immunosorbent assay (ELISA), radioimmunoassay (RIA), immunofluorescent assay (IFA), and immunohistochemistry assay.

8. The method of claim 1, wherein prior to contacting said sample with said antibody, said sample is contacted with α-chymotrypsin.

9. The method of claim 1, wherein said biological sample is selected from the group consisting of blood, urine and synovial fluid.

10. The method of claim 1, wherein said biological sample is blood.

11. A kit for detecting cathepsin K activity in bone, cartilage or soft tissue, said kit comprising an antibody having binding specificity for a neoepitope comprising EAGKPG (SEQ ID NO: 9).

12. The kit of claim 11, wherein said antibody has binding specificity for an epitope comprising GEAGKPG (SEQ ID NO: 1).

13. The kit of claim 11 further comprising, in a separate vial, a label for detecting presence of an immunocomplex formed between said antibody and said neoepitope.

14. The kit of claim 11, wherein said cartilage is human cartilage.

15. The kit of claim 11, wherein presence of cathepsin K activity in cartilage is indicative of cartilage degeneration.

16. The kit of claim 15, wherein said cartilage degeneration is caused by osteoarthritis and/or age associated nonarthritic degeneration.

17. An antibody having binding specificity for a neoepitope comprising EAGKPG (SEQ ID NO: 9).

18. The antibody of claim 17, wherein said antibody has binding specificity for an epitope comprising GEAGKPG (SEQ ID NO: 1).
